(19) United States
(12) Patent Application Publication
(10) Pub. No.: US 2023/0145278 A1
(43) Pub. Date: May 11, 2023

(54) DATA EVALUATION AND ESTIMATION DISPLAY SYSTEM

(71) Applicant: SmileDirectClub LLC, Nashville, TN (US)

(72) Inventors: James Bridgeford, Nashville, TN (US); Philip Harrison, Nashville, TN (US); Lydia Kerr, Nashville, TN (US); Juan Brenes Valverde, Nashville, TN (US); Joseph Cribbs, Nashville, TN (US)

(21) Appl. No.: 17/522,439

(22) Filed: Nov. 9, 2021

(51) Int. Cl. G06F 16/957; G06N 5/02

(52) U.S. Cl. CPC G06F 16/9577; G06F 16/9574; G06N 5/025

(57) ABSTRACT

A system includes instructions stored in memory that cause a processor to collect a first data set, collect a second data set, generate a new data structure using the first data set and the second data set, transmit an electronic data interchange request to a remote third party system, receive an electronic data interchange response containing a third data set, generate a rules engine comprising one or more rules associated with a manufacturer of a device or a service provider and one or more rules associated with a benefit provider, run the rules engine to evaluate the third data set, determine an eligibility status of a user for a product or service, determine a service benefit parameter for the user based on the third data set, determine an obligation parameter, and display the obligation parameter on the user interface of a user device.

### BACKGROUND

Processing user-specific data to determine user-specific benefits and obligations can be time consuming and complex as different parameters may affect the obligation ultimately required to be provided by a particular user. Accordingly, it would be advantageous to employ a rules engine to evaluate unique datasets to more accurately and more quickly determine benefits and obligations for specific users based on collected data sets associated with the specific users.

### SUMMARY

One embodiment relates to a system comprising a processor and a non-transitory computer-readable medium comprising computer-readable instructions stored thereon that when executed by the processor cause the processor to collect a first data set associated with a user by a user interface of a user device, collect a second data set, generate a new data structure using the first data set and the second data set where the new data structure comprises an electronic data interchange request for a third data set, transmit the electronic data interchange request to a remote third party computing system, receive an electronic data interchange response containing the third data set in response to the transmitted electronic data interchange request, determine one or more rules associated with at least one of a manufacturer of a device or a service provider, determine one or more rules associated with a benefit provider, generate a rules engine comprising the one or more rules associated with at least one of the manufacturer or the service provider and the one or more rules associated with the benefit provider that, when enabled, evaluate the third data set based on the first data set and the second data set, run the rules engine to evaluate the third data set using data from at least one of the first data set or the second data set as an input, determine an eligibility status of the user associated with the first data set for at least one of a product or a service provided by at least one of the manufacturer or the service provider based on the evaluation of the third data set by the rules engine, determine a service benefit parameter for the user associated with the first data set based on the evaluation of the third data set by the rules engine, determine an obligation parameter for the user based on the service benefit parameter and the third data set, and display the obligation parameter on the user interface of the user device.

Another embodiment relates to a method comprising collecting a first data set associated with a user by a user interface of a user device, collecting a second data set, generating a new data structure using the first data set and the second data set where the new data structure comprises an electronic data interchange request for a third data set, transmitting the electronic data interchange request to a remote third party computing system, receiving an electronic data interchange response containing the third data set in response to the transmitted electronic data interchange request, determining one or more rules associated with at least one of a manufacturer of a device or a service provider, determining one or more rules associated with a benefit provider, generating a rules engine comprising the one or more rules associated with at least one of the manufacturer or the service provider and the one or more rules associated with the benefit provider that, when enabled, evaluate the third data set based on the first data set and the second data set, running the rules engine to evaluate the third data set using data from at least one of the first data set or the second data set as an input, determining an eligibility status of the user associated with the first data set for at least one of a product or a service provided by at least one of the manufacturer or the service provider based on the evaluation of the third data set by the rules engine, determining a service benefit parameter for the user associated with the first data set based on the evaluation of the third data set by the rules engine, determining an obligation parameter for the user based on the service benefit parameter and the third data set, and displaying the obligation parameter on the user interface of the user device.

Another embodiment relates to a non-transitory computer-readable media comprising computer-readable instructions stored thereon that when executed by a processor cause the processor to collect a first data set, collect a second data set, generate a new data structure using the first data set and the second data set where the new data structure comprises an electronic data interchange request for a third data set, transmit the electronic data interchange request to a remote third party computing system, receive an electronic data interchange response containing the third data set in response to the transmitted electronic data interchange request, determine a service benefit parameter for the user associated with the first data set based on the third data set using a rules engine, identify a plurality of users that share at least one commonality with the user based on the first data set of the user, generate a message based on the service benefit parameter for the user, and provide the message to user devices associated with the plurality of users that share the at least one commonality with the user.

### BRIEF DESCRIPTION OF THE DRAWINGS

FIG. 1A is a data evaluation system configured to evaluate user-specific data, according to an exemplary embodiment.

FIG. 1B is an example user interface of the data evaluation system, according to an exemplary embodiment.

FIG. 2 is another example user interface of the data evaluation system, according to an exemplary embodiment.

FIG. 3 is another example user interface of the data evaluation system, according to an exemplary embodiment.

FIG. 4 is another example user interface of the data evaluation system, according to an exemplary embodiment.

FIG. 5 is another example user interface of the data evaluation system, according to an exemplary embodiment.

FIG. 6 is another example user interface of the data evaluation system, according to an exemplary embodiment.

FIG. 7 is another example user interface of the data evaluation system, according to an exemplary embodiment.

FIG. 8 is another example user interface of the data evaluation system, according to an exemplary embodiment.

FIG. 9 is another example user interface of the data evaluation system, according to an exemplary embodiment.

FIG. 10 is another example user interface of the data evaluation system, according to an exemplary embodiment.

FIG. 11 is another example user interface of the data evaluation system, according to an exemplary embodiment.

FIG. 12 is another example user interface of the data evaluation system, according to an exemplary embodiment.

FIG. 13 is an example flowchart outlining operations of a process for determining a user-specific benefit using the data evaluation system, according to an exemplary embodiment.

FIG. 14A-14D is an example flowchart outlining operations of a process for calculating a user-specific obligation using the data evaluation system, according to an exemplary embodiment.

### DETAILED DESCRIPTION

Following below are more detailed descriptions of various concepts related to, and implementations of, methods, apparatuses, and systems for determining an eligibility status of a user for a benefit and an obligation required to be provided by the user in exchange for a service or product provided to the user. The various concepts introduced above and discussed in greater detail below may be implemented in any number of ways, as the concepts described are not limited to any particular manner of implementation. Examples of specific implementations and applications are provided primarily for illustrative purposes.

Referring to the figures generally, various embodiments disclosed herein relate to systems and methods for evaluating user-specific data and determining an eligibility status of a user for a benefit and an obligation required to be provided by the user in exchange for a service or product provided to the user. The providing of an obligation estimation and benefit estimation to a user can be improved by cutting out a middle man (e.g., professionals, administrative staff, etc.) to directly provide the obligation estimation and benefit estimation to the user directly and immediately. A pricing calculator may automatically evaluate user-specific data (including the user's personal information and policy information) to provide an obligation estimation and benefit estimation directly to the user through a computer application (e.g., a webpage, a mobile app, etc.). In some embodiments, the computer application collects information about the user, analyzes the user's information to determine an obligation estimation and a benefit estimation, then provides the obligation estimation and benefit estimation directly to the user.

While it will be appreciated that the systems and method disclosed herein may be used to determine various types of eligibility statuses for receiving various benefits and for determining various obligations for a user, for ease of reference, examples will be provided specifically relating to determining an eligibility of a patient to receive coverage under an insurance policy for a medical service, and an obligation, or cost, owed by the patient for such service.

For example, insurance coverage may be estimated for medical procedures by analyzing a patient's insurance policy, but the estimation is usually not specific to the service being provided. For example, a typical insurance coverage estimator may determine that a patient has dental insurance that covers preventative dental procedures at 100% of the cost, minor procedures at 80% of the cost, and major procedures at 50% of the cost. However, to determine whether a specific service would be considered a “major procedure” or “a minor procedure” requires a healthcare provider to submit a specific procedure code to the insurance company, which then reviews the code and determines what category it falls into before returning the service categorization to the medical provider, who then presents it to a potential patient. This process may take one or more business days to complete using such typical methods. While waiting for such a process to be completed, a patient may become frustrated by not knowing exactly how much the medical procedure may cost and lose interest in receiving the service or medical device or choose a different healthcare professional to provide the service or medical device to them.

The data evaluation system disclosed herein remedies this problem by collecting patient data, sending patient data and a service codes code to an insurance clearing house, retrieving patient insurance policy information, determining the patient's insurance coverage for the medical service, and determining a cost estimation using a pricing calculator. Additionally, patient data collected by the pricing calculator may be used in the backend to effectively market medical services and devices to other similarly situated patients. For example, as disclosed in further detail below, upon determining that a patient is part of an insurance policy that covers orthodontic procedures, other similarly situated patients can be identified and contacted regarding their eligibility for receiving treatment.

As used herein, the term “medical procedure” is intended to include any medical service and/or medical device provided to a patient by a medical professional, such as a doctor, dentist, or orthodontist. The term “provider” may refer to any entity that provides a benefit to a member, such as an insurance provider that provides insurance coverage to its members. The term “medical provider” may refer to a medical professional that provides a medical procedure, and it may also refer to an entity or organization that provides a medical procedure and/or that may be associated with a network of medical professionals, such as a doctors, dentists, orthodontists, dental service organizations, a dental aligner manufacturer, or any other type of medical professional or entity that provides a medical procedure.

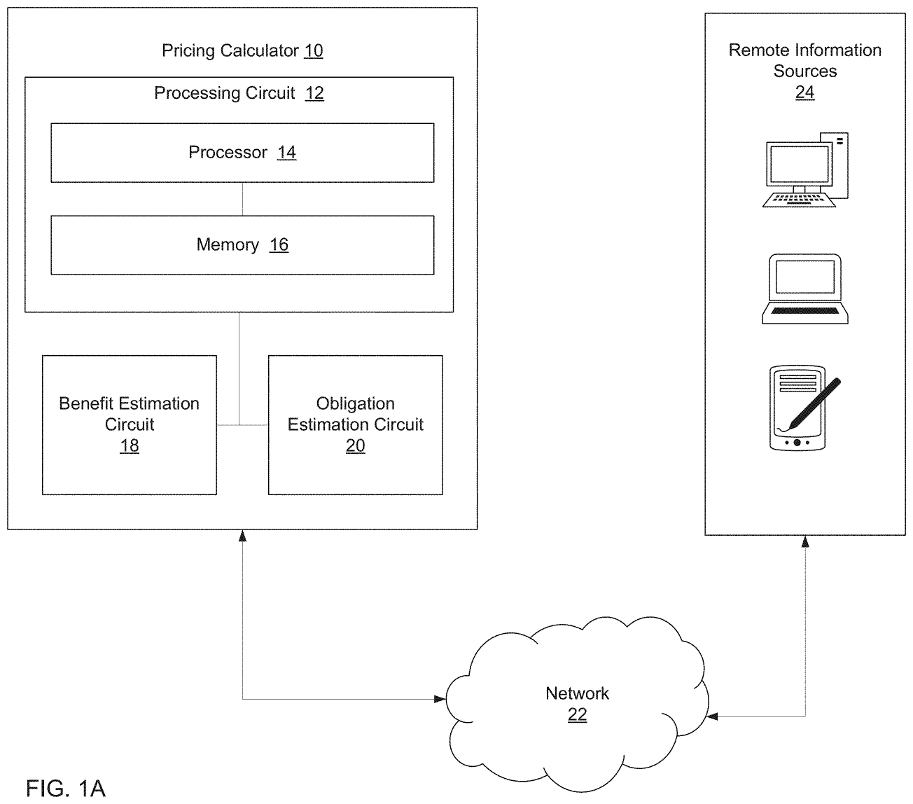

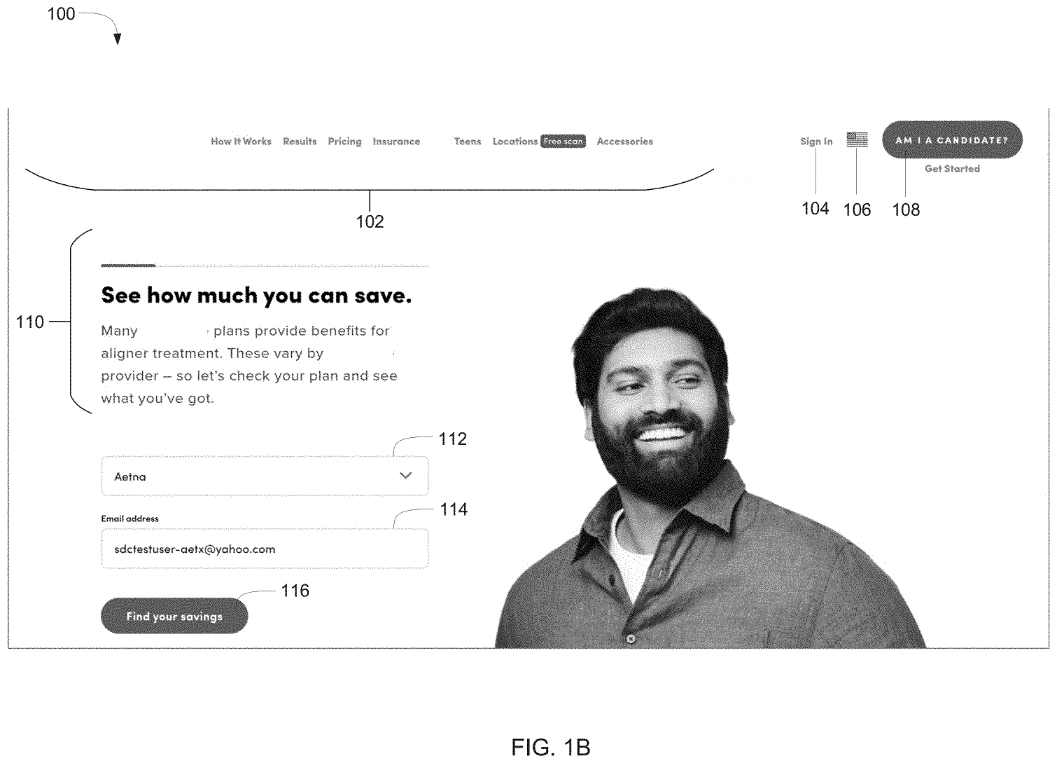

Referring now to FIG. 1A, a data evaluation system including a pricing calculator 10 configured to process user-specific data in order to provide an obligation estimation to a user is shown according to an exemplary embodiment. In some embodiments, pricing calculator 10 includes processing circuit 12 which includes a processor 14 and memory device 16. Additionally, the pricing calculator 10 may include benefit estimation circuit 18 and an obligation estimation circuit 20. The processing circuit 12 may be configured to implement the instructions and/or commands described herein with respect to the benefit estimation circuit 18 and the obligation estimation circuit 20.

In one embodiment, the benefit estimation circuit 18 and the obligation estimation circuit 20 are embodied as machine or computer-readable media storing instructions that are executable by a processor, such as processor 14. As described herein and amongst other uses, the machine-readable media facilitates performance of certain operations to enable reception and transmission of data. For example, the machine-readable media may provide an instruction (e.g., command, etc.) to, e.g., acquire data. In this regard, the machine-readable media may include programmable logic that defines the frequency of acquisition of the data (or, transmission of the data). The computer readable media may include code, which may be written in any programming language including, but not limited to, Java or the like and any conventional procedural programming languages, such as the “C” programming language or similar programming languages. The computer readable program code may be executed on one processor or multiple processors. In the latter scenario, the remote processors may be connected to each other through any type of network (e.g., CAN bus, etc.).

In another embodiment, the benefit estimation circuit 18 and the obligation estimation circuit 20 are embodied as hardware units, such as electronic control units. As such, the benefit estimation circuit 18 and the obligation estimation circuit 20 may be embodied as one or more circuitry components including, but not limited to, processing circuitry, network interfaces, peripheral devices, input devices, output devices, sensors, etc. In some embodiments, the benefit estimation circuit 18 and the obligation estimation circuit 20 may take the form of one or more analog circuits, electronic circuits (e.g., integrated circuits (IC), discrete circuits, system on a chip (SOCs) circuits, microcontrollers, etc.), telecommunication circuits, hybrid circuits, and any other types of “circuit.” In this regard, the benefit estimation circuit 18 and the obligation estimation circuit 20 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The benefit estimation circuit 18 and the obligation estimation circuit 20 may also include programmable hardware devices such as field programmable gate arrays, programmable array logic, programmable logic devices or the like. The benefit estimation circuit 18 and the obligation estimation circuit 20 may include one or more memory devices for storing instructions that are executable by the processor(s) of the benefit estimation circuit 18 and the obligation estimation circuit 20. The one or more memory devices and processor(s) may have the same definition as provided below with respect to the memory 16 and processor 14. In some hardware unit configurations and as described above, the benefit estimation circuit 18 and the obligation estimation circuit 20 may be geographically dispersed throughout separate locations in the pricing calculator 10. Alternatively and as shown, the benefit estimation circuit 18 and the obligation estimation circuit 20 may be embodied in or within a single unit/housing, which is shown as the pricing calculator 10.

In the example shown, the pricing calculator 10 includes the processing circuit 12 having a processor 14 and a memory 16. The processing circuit 12 may be configured to execute or implant the instructions, commands, and/or control processes described herein with respect to the benefit estimation circuit 18 and the obligation estimation circuit 20. The depicted configuration represents the benefit estimation circuit 18 and the obligation estimation circuit 20 as machine or computer-readable media. However, as mentioned above, this illustration is not meant to be limiting as the present disclosure contemplates other embodiments where the benefit estimation circuit 18 and the obligation estimation circuit 20, or at least one circuit of the benefit estimation circuit 18 and the obligation estimation circuit 20, is configured as a hardware unit. All such combinations and variations are intended to fall within the scope of the present disclosure.

The processor 14 may be implemented as one or more processors, one or more application specific integrated circuits (ASIC), one or more field programmable gate arrays (FPGAs), a digital signal processor (DSP), a group of processing components, or other suitable electronic processing components. The one or more processors may be shared by multiple circuits (e.g., the benefit estimation circuit 18 and the obligation estimation circuit 20 may comprise or otherwise share the same processor which, in some example embodiments, may execute instructions stored, or otherwise accessed, via different areas of memory). Alternatively or additionally, the one or more processors may be configured to perform or otherwise execute certain operations independent of one or more co-processors. In other example embodiments, two or more processors may be coupled via a bus to enable independent, parallel, pipelined, or multi-threaded instruction execution. All such variations are intended to fall within the scope of the present disclosure. The memory 16 (e.g., RAM, ROM, Flash Memory, hard disk storage, etc.) may store data and/or computer code for facilitating the various processes described herein. The memory 16 may be communicably coupled to the processor 14 to provide computer code or instructions to the processor 14 for executing at least some of the processes described herein. Moreover, the memory 16 may be or include tangible, non-transient volatile memory or non-volatile memory. Accordingly, the memory 16 may include database components, object code components, script components, or any other type of information structure for supporting the various activities and information structures described herein.

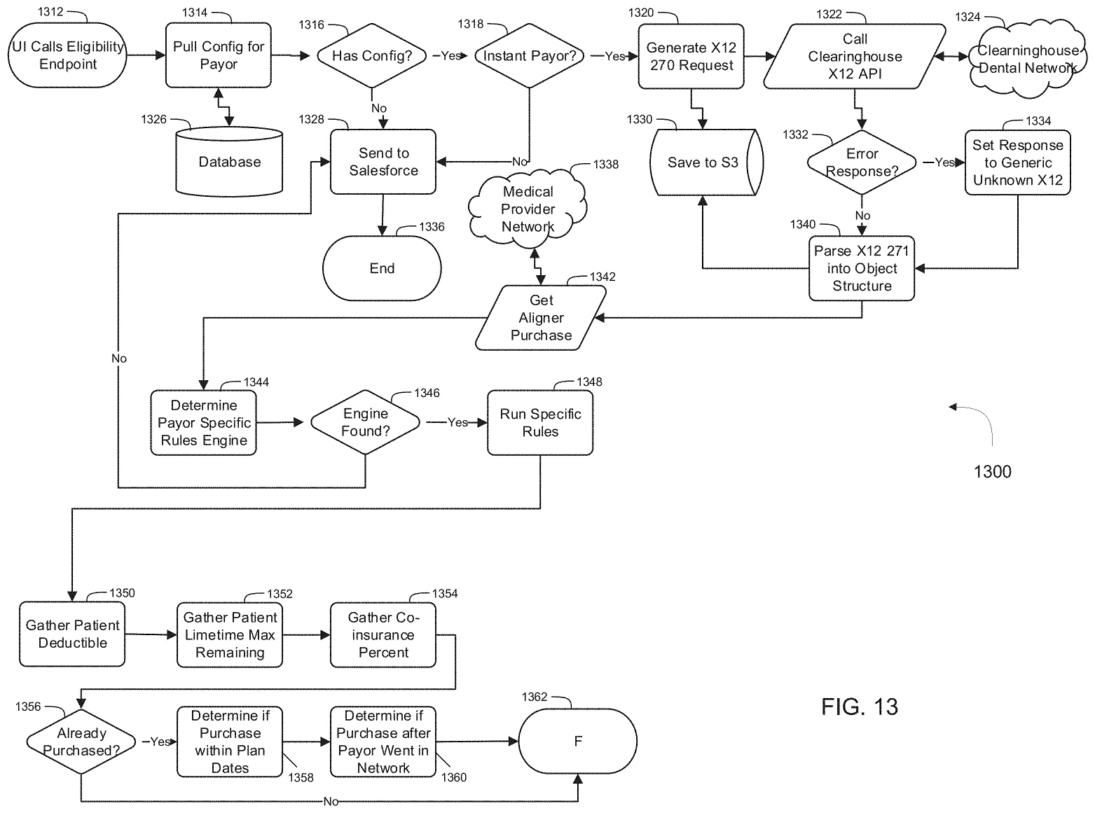

The benefit estimation circuit 18 is configured to determine the coverage for a specialized medical procedure for a particular user. As shown in FIGS. 1-12 below, user data (e.g., the user's personal and insurance information) may be collected through remote information sources 24. Remote information sources 24 can be any type of computing devices (e.g., mobile devices, laptops, desktop computers, cell phones, etc.) in which a user may input information. Once the user enters their data through the remote information source, this data may be communicated over the network 22 to the pricing calculator 10 for further processing. The process for determining the coverage for a specialized medical procedure for a particular user is explained in more detail with respect to FIG. 13. The obligation estimation circuit 20 is configured to calculate an obligation estimation or price for a specialized medical procedure for a particular user based on the original price of the procedure minus any coverage and/or discounts.

Referring now to FIG. 1B, an example user interface generated by the pricing calculator 10 is shown, according to an exemplary embodiment. A user may be interested in a medical procedure such as teeth alignment, but may also want to explore pricing options before settling on a service. In some embodiments, user interface 100 is the first page of a series of user interfaces that collect user information that may be used by a pricing calculator to determine an obligation estimation for receiving a medical service, such as dental aligner therapy. In some embodiments, user interface 100 may include a main site menu 102 configured to provide easy navigation of the website for the user. In some embodiments, the main site menu 102 may include one or more menu items (e.g., “How It Works”, “Results”, “Pricing”, “Insurance”, “Teens”, “Locations”, “Free Scans”, and “Accessories”. In some embodiments, a user may access different parts of a medical provider's website by selecting the menu items. In some embodiments, the user interface 100 may also include a sign in button 104 configured to enable a user to sign into the website (e.g., if the user already has an account). In some embodiments, the user interface 100 includes a select region button 106 configured to allow a user to select which geographical region they are located and/or which language they would like to view the website in. In some embodiments, the user interface 100 includes a get started button 108 that directs the user to an introductory product and/or service landing page. In some embodiments, the user interface 100 may include a promotional banner that may present promotional codes for a customer (e.g., such as a discount).

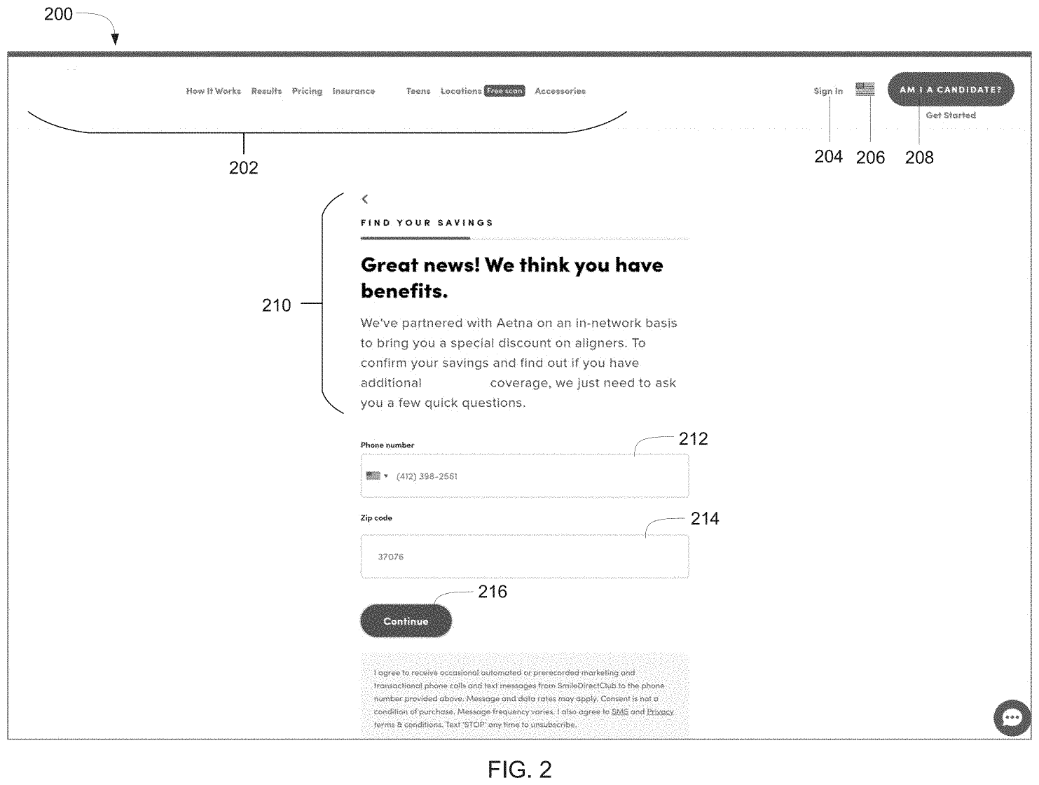

The user interface 100 is configured to collect a user's information at interface portions 112 and 114. In some embodiments, the user interface 100 may include a page description 110 that describes the type of information the webpage is requesting and why the information is requested. For example, user interface 100 includes a description stating that the user may save money through their coverage. In some embodiments, the user may enter their provider at interface portion 112. In some embodiments, the user may enter their email address at interface portion 114. Once the user has entered their information (e.g., user's provider and email), the user can proceed to the second page of the series of user interfaces by selecting the continuation button 116. The second page of the series of user interfaces is described in more detail with respect to FIG. 2.

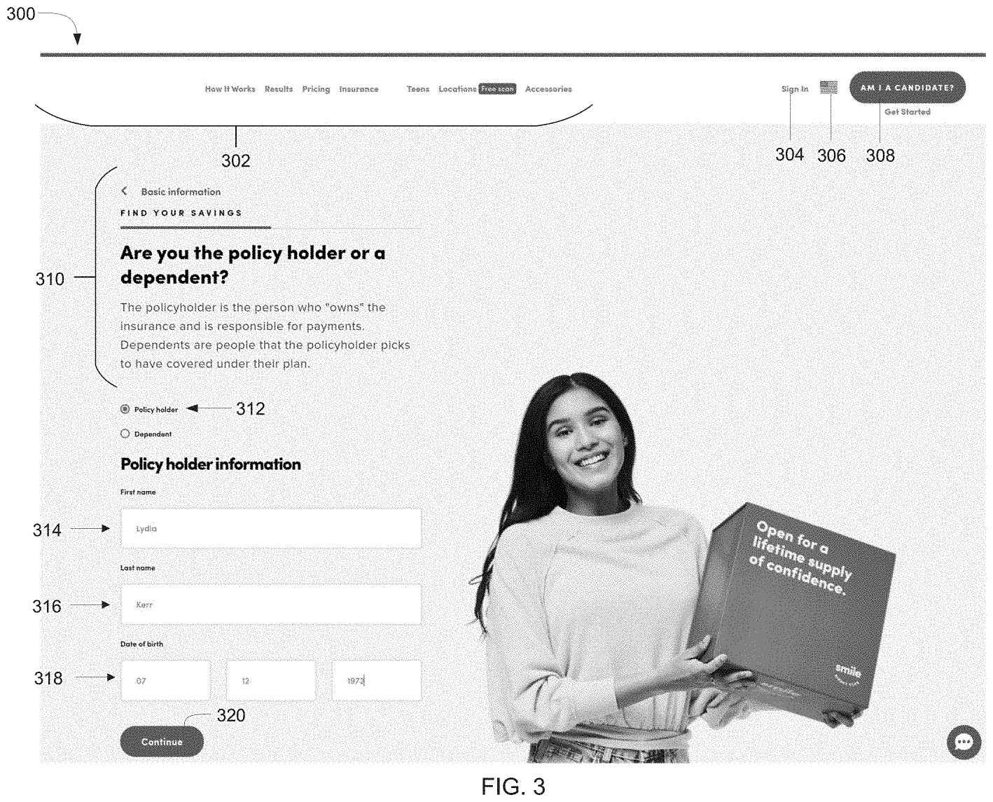

Referring to FIG. 2, an example user interface showing a second page of a series of user interfaces is shown, according to an exemplary embodiment. In some embodiments, user interface 200 is the second page of a series of user interfaces that collect user data to be evaluated by the pricing calculator 10 in order to determine an obligation estimation for a user. The user interface 200 includes many elements similar to the elements described in user interface 100. For example, the user interface 200 includes main menu 202 similar to main menu 102 and buttons 204, 206, and 208 similar to buttons 104, 106, and 108 described above. Additionally, the user interface 200 includes a page description 210 similar to page description 110. In this case, the page description 210 describes that the medical provider partners with the users provider and the user may have access to special discounts and in-network rates. Before an obligation estimation is provided to the user, more information about the user needs to be collected which the user may enter at user interface portions 212 and 214. In some embodiments, the user may enter their phone number at user interface portion 212. In some embodiments, the user may enter their zip code at user interface portion 214. Once the user has entered this information, they may continue to the third page of the series of user interfaces by selecting the continue button 216. The third page of the series of user interfaces is described in more detail with respect to FIG. 3.

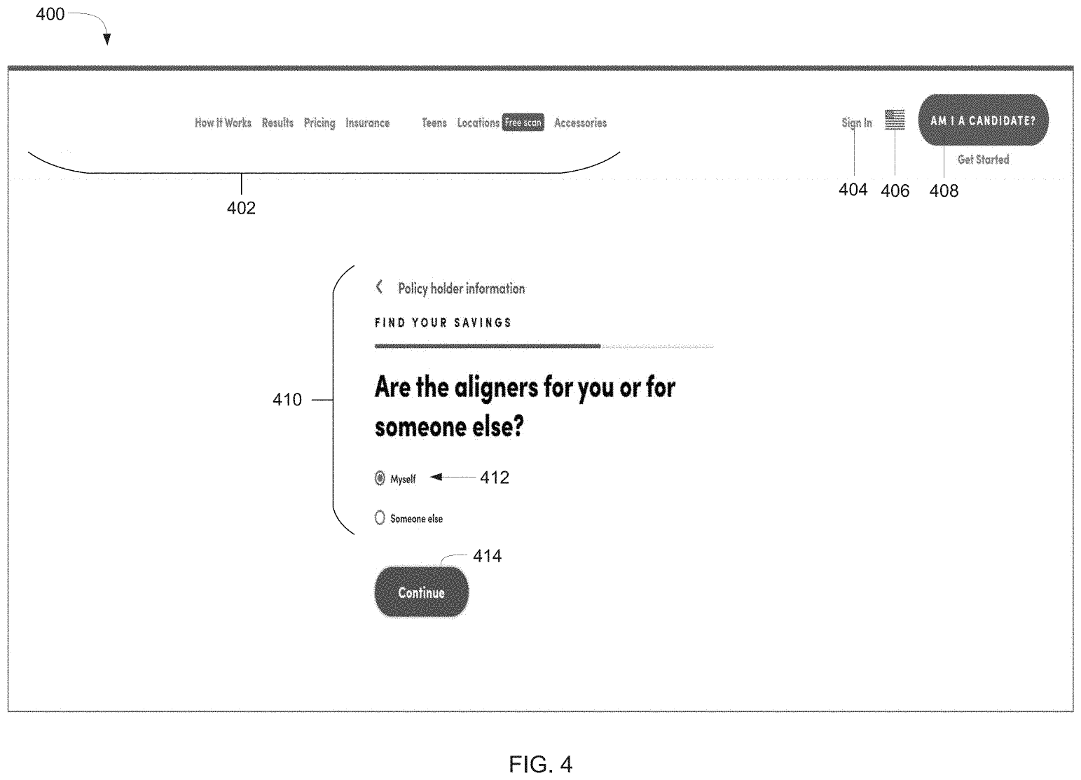

Referring to FIG. 3, an example user interface showing a third page of a series of user interfaces is shown, according to an exemplary embodiment. In some embodiments, user interface 300 is the third page of a series of user interfaces that collects user information that may be used by a pricing calculator to determine an obligation estimation for receiving a medical service, such as dental aligner therapy. The user interface 300 includes many elements similar to the elements described in user interface 100. For example, the user interface 300 includes main menu 302 similar to main menu 102 and buttons 304, 306, and 308 similar to buttons 104, 106, and 108 described above. Additionally, the user interface 300 includes a page description 310 similar to page description 110. In this case, the page description 310 asks the user if they are a policy holder or a dependent. In the example shown in FIGS. 1-6, the user filling out the form is the policy holder. At user interface portion 312, the user can input whether they are the policy holder or the dependent. At user interface portions 314-318, the user can input their (e.g., the policy holder's) information including their first name, their last name, and their date of birth. Once the user has entered this information, they may continue to the fourth page of the series of user interfaces by selecting the continue button 320. The fourth page of the series of user interfaces is described in more detail with respect to FIG. 4.

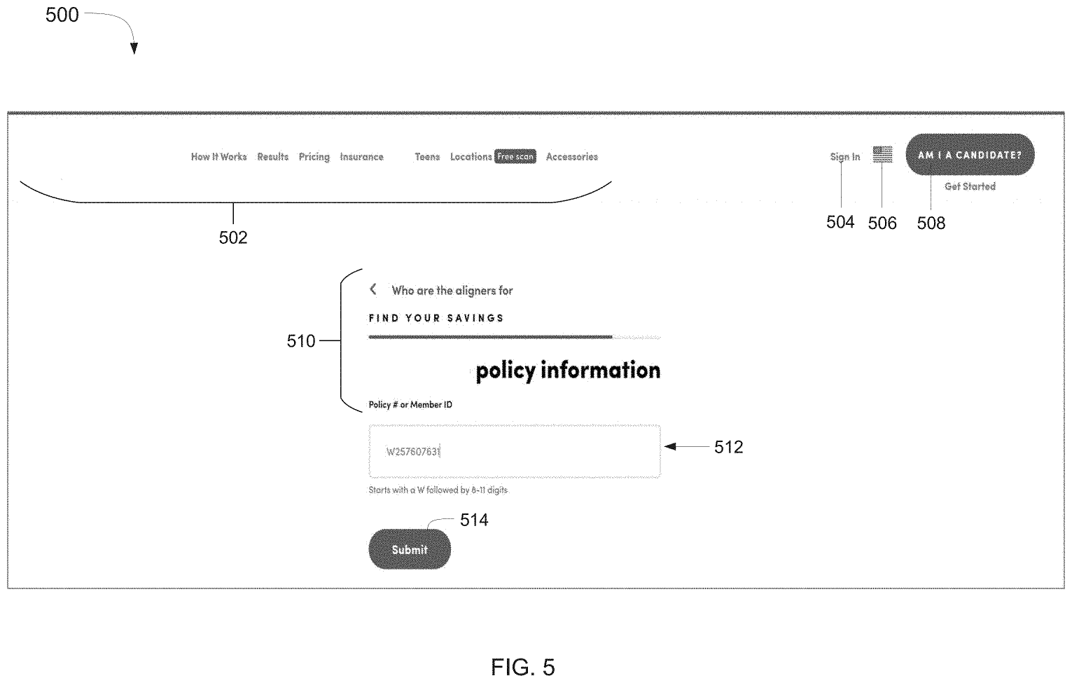

Referring to FIG. 4, an example user interface showing a fourth page of a series of user interfaces is shown, according to an exemplary embodiment. In some embodiments, user interface 400 is the fourth page of a series of user interfaces that collects user information that may be used by a pricing calculator to determine an obligation estimation for receiving a medical service, such as dental aligner therapy. The user interface 400 includes many elements similar to the elements described in user interface 100. For example, the user interface 400 includes main menu 402 similar to main menu 102 and buttons 404, 406, and 408 similar to buttons 104, 106, and 108 described above. Additionally, the user interface 400 includes a page description 410 similar to page description 110. In this case, the page description 410 asks the user if they are purchasing dental aligners for themselves or someone else. At user interface portion 412, the user can input whether the aligners are for themselves or someone else. Once the user has made their selection, they may continue to the fifth page of the series of user interfaces by selecting the continue button 414. The fifth page of the series of user interfaces is described in more detail with respect to FIG. 5.

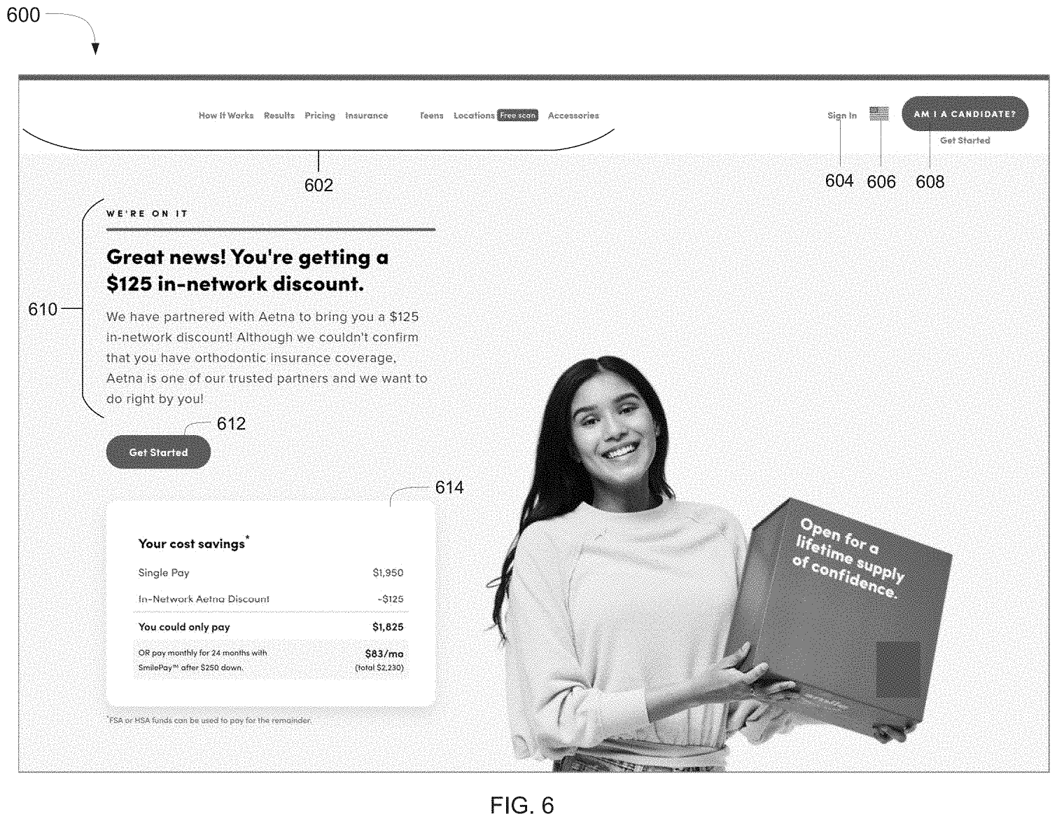

Referring to FIG. 5, an example user interface showing a fifth page of a series of user interfaces is shown, according to an exemplary embodiment. In some embodiments, user interface 500 is the fifth page of a series of user interfaces that collects user information that may be used by a pricing calculator to determine an obligation estimation for receiving a medical service, such as dental aligner therapy. The user interface 500 includes many elements similar to the elements described in user interface 100. For example, the user interface 500 includes main menu 502 similar to main menu 102 and buttons 504, 506, and 508 similar to buttons 104, 106, and 108 described above. Additionally, the user interface 500 includes a page description 510 similar to page description 110. In this case, the page description 510 asks the user for their insurance policy information (e.g., a policy number or member ID). At user interface portion 512, the user can input their policy number or member ID. Once the user has entered this information, they may continue to the sixth and final page of the series of user interfaces by selecting the continue button 514. The sixth page of the series of user interfaces is described in more detail with respect to FIG. 6.

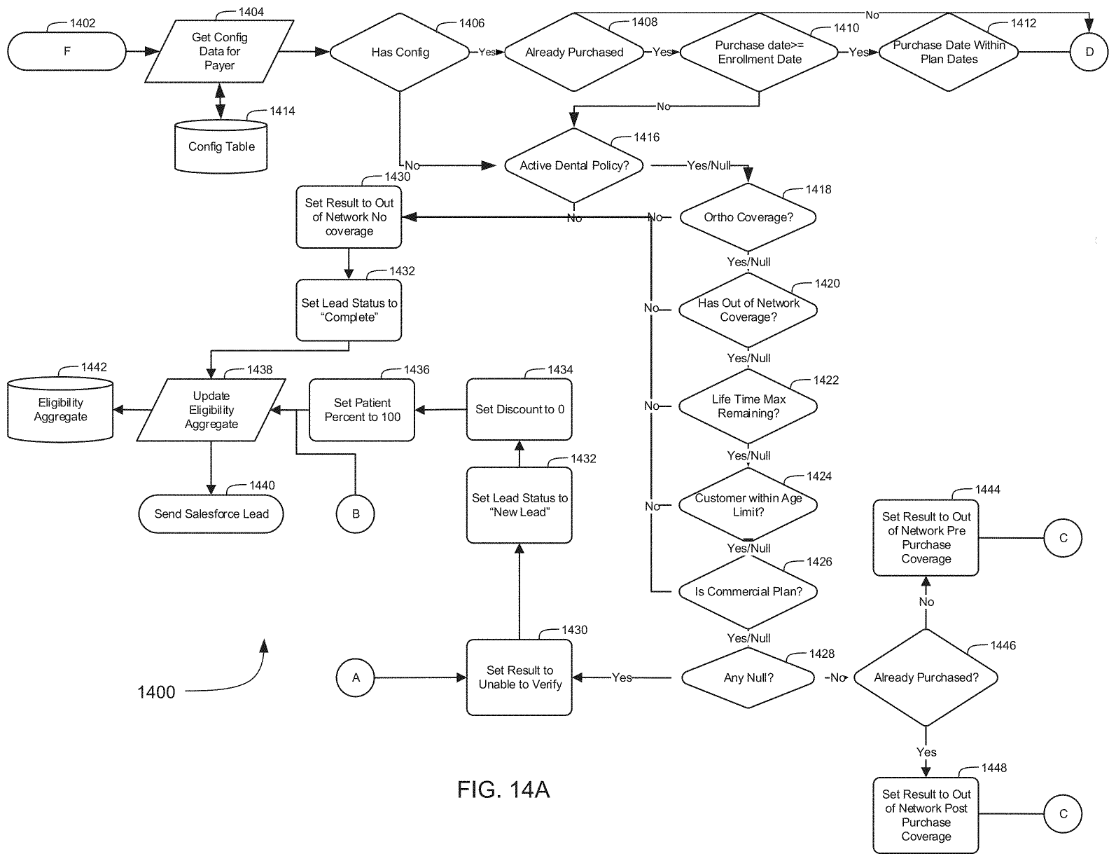

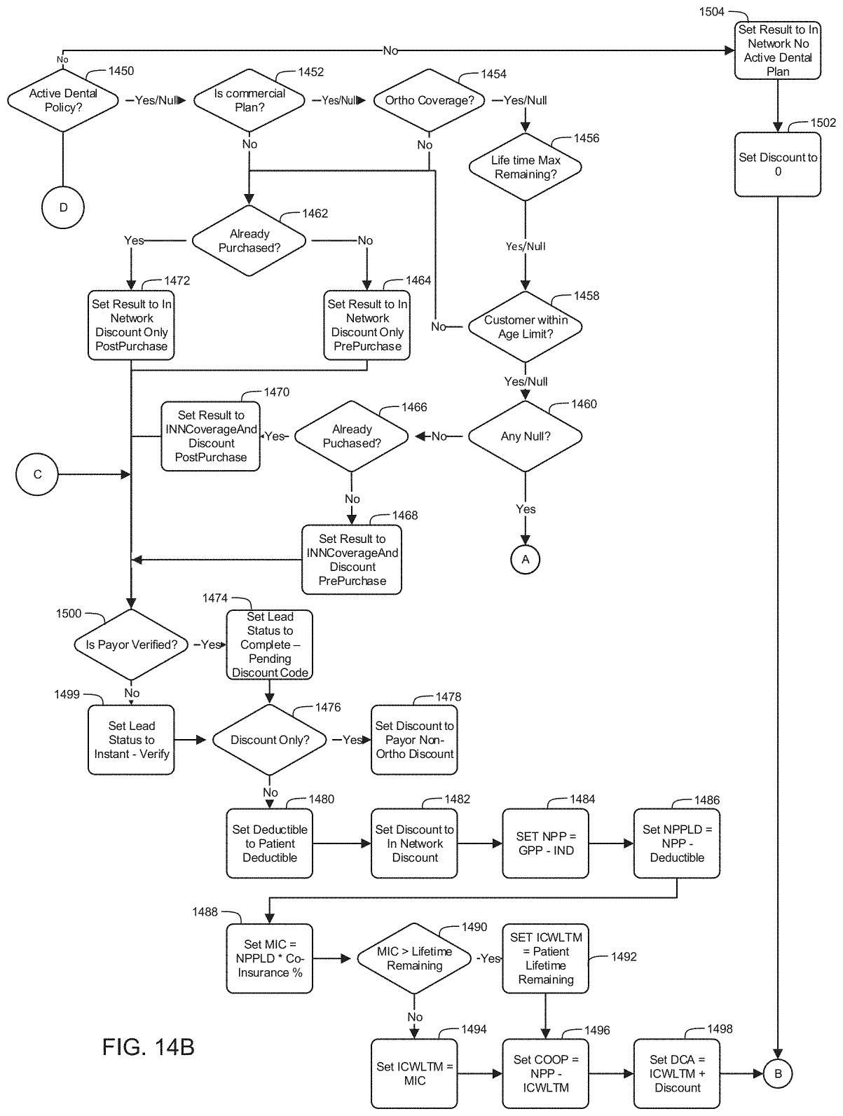

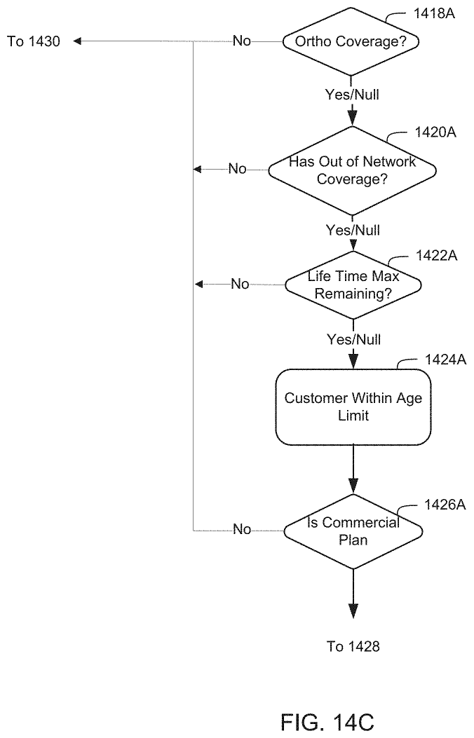

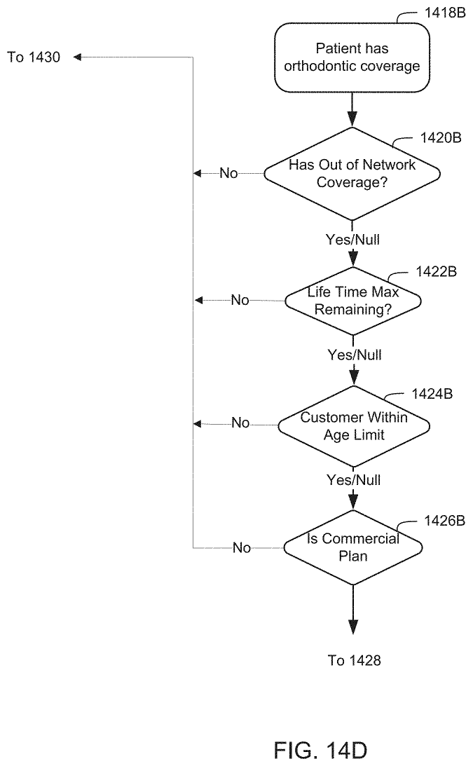

Referring to FIG. 6, an example user interface showing a sixth page of a series of user interfaces is shown, according to an exemplary embodiment. In some embodiments, user interface 600 is the sixth page of a series of user interfaces that collects user information that may be used by a pricing calculator to determine an obligation estimation for receiving a medical service, such as dental aligner therapy. User interface 600 may be configured to display the cost estimation and benefit estimation to a user. The user interface 600 includes many elements similar to the elements described in user interface 100. For example, the user interface 600 includes main menu 602 similar to main menu 102 and buttons 604, 606, and 608 similar to buttons 104, 106, and 108 described above. Additionally, the user interface 600 includes a page description 610 similar to page description 110. In this case, the page description 610 informs the user that they have an in-network discount. User interface portion 614 is configured to provide an obligation estimation to the user. For example, the user interface portion includes the total cost before discount (e.g., $1950), the in-network insurance discount ($125), estimated total cost for the user (e.g., $1825), and a breakdown of what it would cost the user on a monthly basis (e.g., $83), and the total cost should the user elect to pay monthly (e.g., $2230). After analyzing the cost estimation provided in user interface 600, the user may choose to continue with the medical procedure by selecting the continue button 614. The process for determining the benefit estimation and obligation estimation is described in more detail with respect to FIGS. 13-14D.

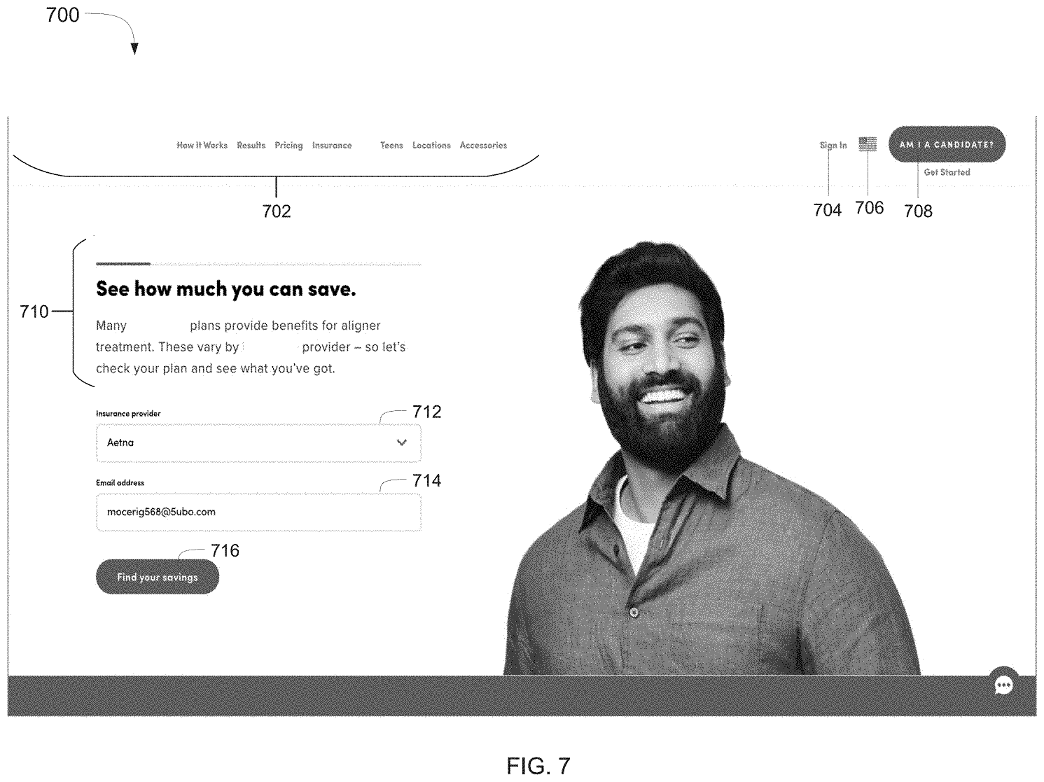

FIGS. 7-12 describe a similar series of user interfaces described in FIGS. 1-6, but differs in the aspect that is for a dependent of a policy holder as opposed to the actual policy holder themselves. Referring now to FIG. 7, an example user interface showing a first page of a series of user interfaces is shown, according to an exemplary embodiment. A user may be interested in a medical procedure such as teeth alignment, but may also want to explore pricing options before settling on a service. In some embodiments, user interface 700 is the first page of a series of user interfaces that collects user information that may be used by a pricing calculator to determine an obligation estimation for receiving a medical service, such as dental aligner therapy. In some embodiments, user interface 700 may include a main site menu 702 configured to provide easy navigation of the website for the user. In some embodiments, the main site menu 102 may include one or more menu items (e.g., “How It Works”, “Results”, “Pricing”, “Insurance”, “Teens”, “Locations”, “Free Scans”, and “Accessories”. In some embodiments, a user may access different parts of a medical provider's website by selecting the menu items. In some embodiments, the user interface 700 may also include a sign in button 704 configured to enable a user to sign into the website (e.g., if the user already has an account). In some embodiments, the user interface 700 includes a select region button 706 configured to allow a user to select which geographical region they are located. In some embodiments, the user interface 700 includes a get started button 708 that directs the user to an introductory product and/or service landing page.

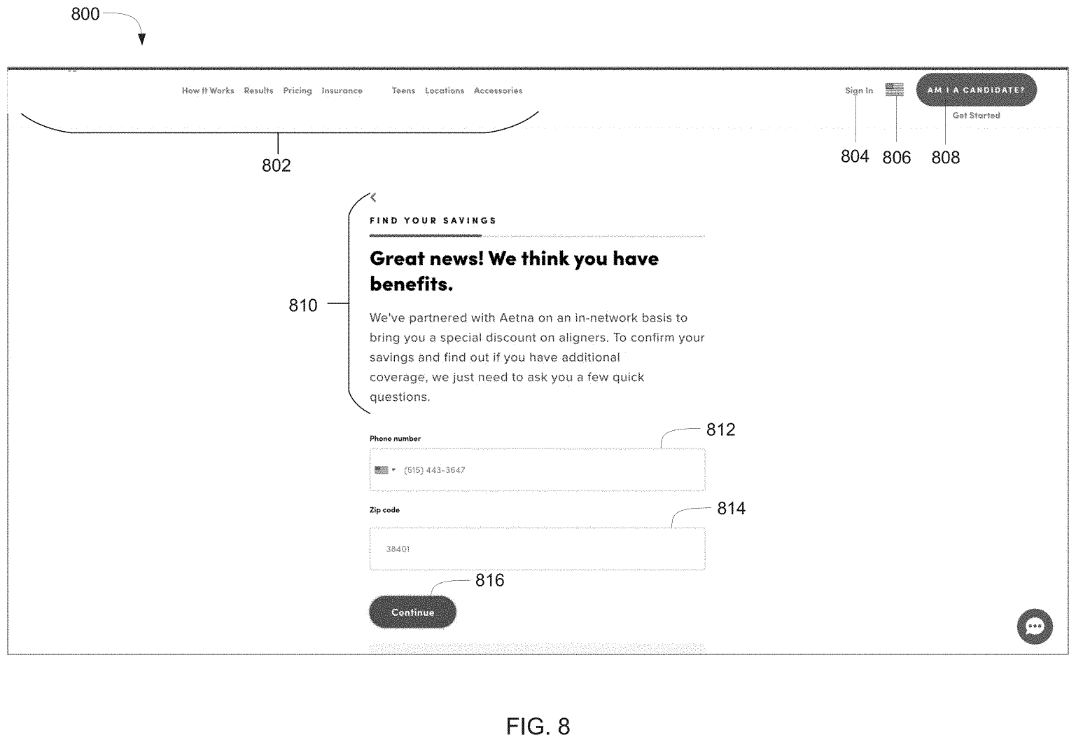

The user interface 700 is configured to collect a user's information at interface portions 712 and 714. In some embodiments, the user interface 700 may include a page description 710 that describes the type of information the webpage is requesting and why the information is requested. For example, user interface 700 includes a description describing that the user may save money through their coverage. In some embodiments, the user may enter their provider at interface portion 712. In some embodiments, the user may enter their email address at interface portion 714. Once the user has entered their information (e.g., user's provider and email), the user can proceed to the second page of the series of user interfaces by selecting the continuation button 716. The second page of the series of user interfaces is described in more detail with respect to FIG.8.

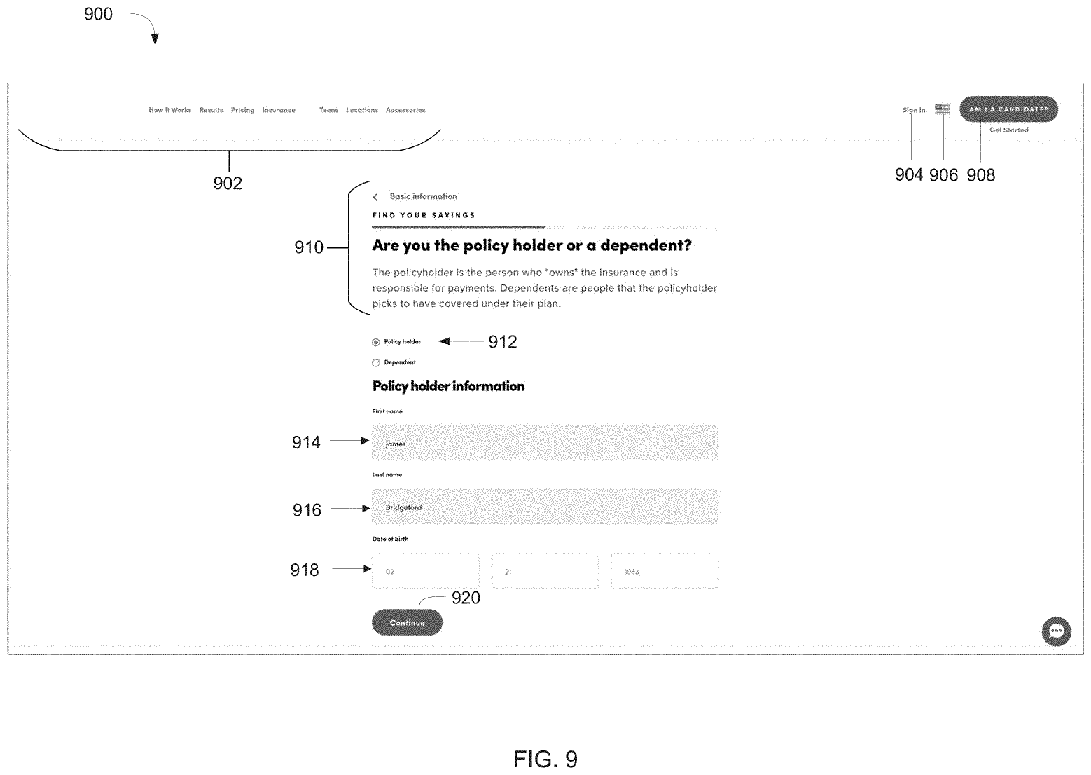

Referring to FIG. 8, an example user interface showing a second page of a series of user interfaces is shown, according to an exemplary embodiment. In some embodiments, user interface 800 is the second page of a series of user interfaces that collects user information that may be used by a pricing calculator to determine an obligation estimation for receiving a medical service, such as dental aligner therapy. The user interface 800 includes many elements similar to the elements described in user interface 700. For example, the user interface 800 includes main menu 802 similar to main menu 702 and buttons 804, 806, and 808 similar to buttons 704, 706, and 708 described above. Additionally, the user interface 800 includes a page description 810 similar to page description 710. In this case, the page description 810 describes that the medical provider partners with the user's provider and the user may have access to special discounts and in-network rates. Before an obligation estimation is provided to the user, more information about the user needs to be collected which the user may enter at user interface portions 812 and 814. In some embodiments, the user may enter their phone number at user interface portion 812. In some embodiments, the user may enter their zip code at user interface portion 814. Once the user has entered this information, they may continue to the third page of the series of user interfaces by selecting the continue button 816. The third page of the series of user interfaces is described in more detail with respect to FIG. 9.

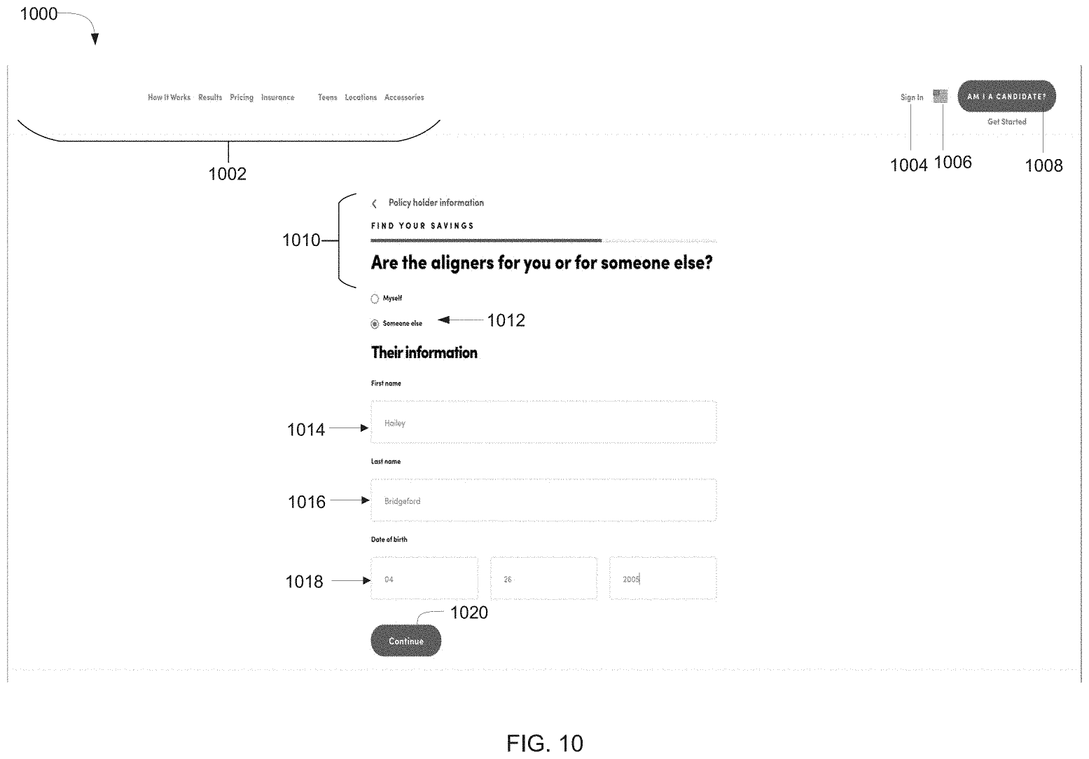

Referring to FIG. 9, an example user interface showing a third page of a series of user interfaces is shown, according to an exemplary embodiment. In some embodiments, user interface 900 is the third page of a series of user interfaces that collects user information that may be used by a pricing calculator to determine an obligation estimation for receiving a medical service, such as dental aligner therapy. The user interface 900 includes many elements similar to the elements described in user interface 700. For example, the user interface 900 includes main menu 902 similar to main menu 702 and buttons 904, 906, and 908 similar to buttons 704, 706, and 708 described above. Additionally, the user interface 900 includes a page description 910 similar to page description 710. In this case, the page description 910 asks the user if they are a policy holder or a dependent. At user interface portion 912, the user can input whether they are the policy holder or the dependent. At user interface portions 914-918, the user can input their (e.g., the policy holder's) information including their first name, their last name, and their date of birth. Once the user has entered this information, they may continue to the fourth page of the series of user interfaces by selecting the continue button 920. The fourth page of the series of user interfaces is described in more detail with respect to FIG. 10.

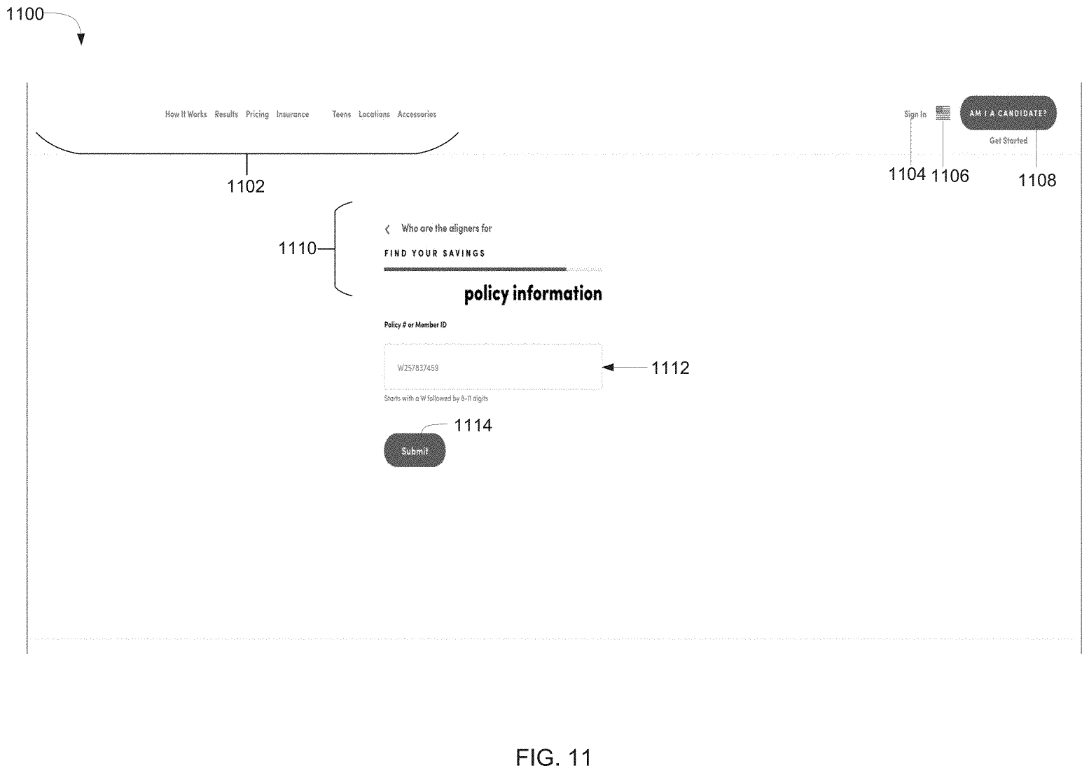

Referring to FIG. 10, an example user interface showing a fourth page of a series of user interfaces is shown, according to an exemplary embodiment. In some embodiments, user interface 1000 is the fourth page of a series of user interfaces that collects user information that may be used by a pricing calculator to determine an obligation estimation for receiving a medical service, such as dental aligner therapy. The user interface 1000 includes many elements similar to the elements described in user interface 700. For example, the user interface 1000 includes main menu 1002 similar to main menu 702 and buttons 1004, 1006, and 1008 similar to buttons 704, 706, and 708 described above. Additionally, the user interface 1000 includes a page description 1010 similar to page description 710. In this case, the page description 1010 asks the user if they are purchasing dental aligners for themselves or someone else. At user interface portion 1012, the user can input whether the aligners are for themselves or someone else. In this case, the user has selected that the aligners are for someone else. At user interface portions 1014 — 1018, the user may enter the other person's first name, last name, and date of birth. Once this information has been entered, the user may continue to the fifth page of the series of user interfaces by selecting the continue button 1020. The fifth page of the series of user interfaces is described in more detail with respect to FIG. 11.

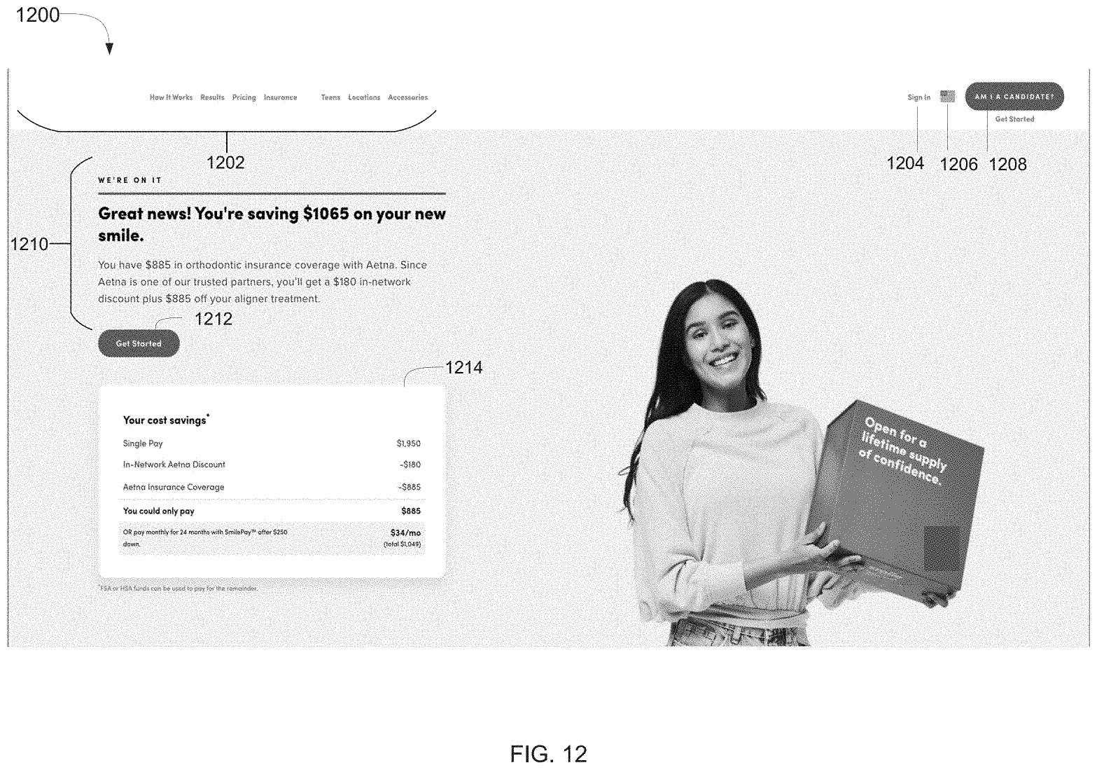

Referring to FIG. 11, an example user interface showing a fifth page of a series of user interfaces is shown, according to an exemplary embodiment. In some embodiments, user interface 1100 is the fifth page of a series of user interfaces that collects user information that may be used by a pricing calculator to determine an obligation estimation for receiving a medical service, such as dental aligner therapy. The user interface 1100 includes many elements similar to the elements described in user interface 700. For example, the user interface 1100 includes main menu 1102 similar to main menu 702 and buttons 1104, 1106, and 1108 similar to buttons 704, 706, and 708 described above. Additionally, the user interface 1100 includes a page description 1110 similar to page description 710. In this case, the page description 1110 asks the user for their insurance policy information (e.g., a policy number or member ID). At user interface portion 1112, the user can input their policy number or member ID. Once the user has entered this information, they may continue to the sixth and final page of the series of user interfaces by selecting the continue button 1114. The sixth page of the series of user interfaces is described in more detail with respect to FIG. 12.

Referring to FIG. 12, an example user interface showing a sixth page of a series of user interfaces is shown, according to an exemplary embodiment. In some embodiments, user interface 1200 is the sixth page of a series of user interfaces that collects user information that may be used by a pricing calculator to determine an obligation estimation for receiving a medical service, such as dental aligner therapy. User interface 1200 may be configured to display the cost estimation and benefit estimation to a user. The user interface 1200 includes many elements similar to the elements described in user interface 700. For example, the user interface 1200 includes main menu 1202 similar to main menu 702 and buttons 1204, 1206, and 1208 similar to buttons 704, 706, and 708 described above. Additionally, the user interface 1200 includes a page description 1210 similar to page description 710. In this case, the page description 1210 informs the user that they have an in-network discount and orthodontic insurance coverage. User interface portion 1214 is configured to provide an obligation estimation to the user. For example, the user interface portion 1214 includes the total cost before discount (e.g., $1950), the in-network insurance discount ($180), the coverage amount ($885), estimated total cost for the user (e.g., $885), and a breakdown of what it would cost the user on a monthly basis (e.g., $34), and the total cost should the user elect to pay monthly (e.g., $1049). After analyzing the cost estimation provided in user interface 1200, the user may choose to continue with the medical procedure by selecting the continue button 1212. The process for determining the benefit estimation and obligation estimation is described in more detail with respect to FIGS. 13-14D.

FIGS. 13-14D describe the processes for determining a user's coverage and cost estimation for a medical procedure. More specifically, the processes described with respect to FIGS. 13-14D enable professionals to automate the benefit estimation and obligation/cost estimation for providing a medical procedure, such as providing dental aligner therapy. In FIGS. 13-14D, the term “True” signifies a passed validation, the term “False” signifies a failed validation, the term “Null” signifies that a condition is unable to be verified, “NPP” means “Net Product Price,” NPPLD means “Net Product Price Less Deductible,” “ICWLTM” means “Insurance Coverage With Lifetime Max,” “COOP” means “Customer Out Of Pocket,” “DCA” means “Discount Code Amount,” “GPP” means “Gross Product Price,” “IND” means “In Network Discount,” “NOD” means “Non-Ortho Discount,” and “MIC” means “Max Insurance Coverage.”

Referring now to FIG. 13, a process 1300 for processing a user's insurance information and determining a user's benefit or coverage is shown, according to an exemplary embodiment. The process 1300 can be performed by one or more processors, such as processor 14. Before beginning process 1300, user data (e.g., the user's personal and insurance information) is collected through a user interface from remote information sources 24 as shown in FIGS. 1-12. As mentioned above, the user's personal information and insurance information can include but is not limited to the user's name, the user's provider, the user's phone number, the user's zip code, the user's date of birth, the user's policy number and/or member ID, etc. Once the user data has been collected, the process 1300 begins at operation 1312. At operation 1312, the user interface calls the eligibility endpoint signaling that the user data has been collected and is ready to be processed. At operation 1314, the pricing calculator 10 may pull a configuration for the payor in order to process the user data. The medical provider may use one or more APIs (e.g., one API, two APIs, etc.) to pull payor configurations based on one or more payor identifiers, including payor name, payor mnemonic, payor discount, etc. For example, the medical provider may use one or more AWS APIs. Pulling the configuration for a payor may be defined as looking up the payor in an internal system to see if the payor has a set of configuration data stored. In some embodiments, the internal system may be memory 16 which may store payor configurations. The payor configuration may be a set of information about the payor. If the payor has a configuration, it is pulled before being used in the rest of the process 1300. In some embodiments, the processor may pull a configuration from an external database (e.g., database 1326).

At operation 1316, the pricing calculator 10 determines whether the user data has an associated configuration that has been pulled. If the user data does not have a configuration available to be pulled, the user's data is sent to a third party customer handling software 1328 and the process 1300 ends at operation 1336. In some embodiments, the information sent to the third party customer handling software (e.g., such as software provided by Salesforce) may be used for purposes of marketing the medical provider's services. For example, if the medical provider determines that an insurance policy associated with a certain employer covers treatments provided by the medical provider (e.g., dental aligner therapy for straightening teeth), the medical provider may infer that other employees employed by that employer have similar coverage and therefore present targeted advertisements to other employees employed by that employer. The advertisements may further be targeted to similarly situated employees of the employer, such as other employees of the employer of similar age (e.g., the same age, born in the same year, born within 2 years, above or below an age threshold such as 12 years of age or 16 years or age or 18 years of age, or 25 years of age) or other employees of the employer with similar professional titles (e.g. executives, support staff, associates, etc.) because employees at different professional levels may receive different benefits. The advertisements may further be targeted to family members of other employees of the employer, such as children of the employees or spouses or partners of the employees.

In some embodiments, potential user information used to target advertisements may be obtained from data collected on social media sites. For example, employee information including employer and professional title may be obtained from professional social media sites such as Linkedln. Additionally, family member information may be obtained from a variety of social media sites such as Facebook. Social media may also be used to distribute targeted advertisements for potential users. For example, a medical provider may purchase advertisements (e.g., promoted posts, banner advertisements, or any other type of advertisements) on a professional social media site targeting professionals employed by a specific employer. In another example, a medical provider may purchase advertisements on a social media site targeting family members of a certain employee or family members of a certain type of employee (e.g., an employee with the same or a similar level or type of position).

More specifically, data received from social media sites that sell user data (e.g., user name, user age, address, employer, familial status, etc.) to potential advertisers can be used by the medical provider to determine that a particular person works at a particular company that enrolls all their employees in an insurance plan that covers child (e.g., persons 14 years or younger, persons 16 years or younger, persons 18 years or younger) orthodontic procedures at 100% of cost. Further using the user data obtained from a social media site, the medical provider may determine that a particular person has two children under the age of 14. Given this information, the medical provider may create a targeted advertisement configured to inform that particular person that their child's orthodontic treatment may be covered at 100% with the medical provider. Additionally, this targeted advertisement may be displayed to that particular person through the social media site or through another application or website.

In another example, a social media site may also sell user data (e.g., user name, user age, address, employer, familial status, etc.) to potential advertisers. Using this user data, the medical provider may determine that a particular company enrolls all their executive-level employees in an insurance plan that covers all adult orthodontic procedures at 50%. The medical provider may create a targeted advertisement for all executive-level employees for the particular company that lets the employees know that they may qualify for 50% off an orthodontic procedure provided by the medical provider. The medical provider may also distribute this advertisement on the social media site, another social media site, or another website.

If the user data has a configuration available within the database, the process 1300 continues to operation 1318. At operation 1318, the pricing calculator 10 determines if the user's provider is an instant payor. In some embodiments, an instant payor may be defined as a provider that has partnered with medical provider. If the user is determined to not be an instant payor, the user's information is sent to a third party customer handling software 1328 and the process 1300 ends at operation 1336. If the user is determined to be an instant payor, the process 1300 continues to operation 1320.

At operation 1320, the pricing calculator 10 generates an X12 270 request that may be sent to an insurance network in order to determine insurance policy details for a particular user. In some embodiments, the X12 270 request may be defined as electronic data interchange (EDI) transaction in which private and confidential healthcare information for a user may be securely transmitted to an insurance network in order to estimate the coverage a user could have for a medical procedure. The X12 270 request is saved to the S3 database. The S3 database is a file storing database that stores both the X12 270 request generated at operation 1320 and the request response generated at operation 1340 for further review if necessary. For example, if a payor wishes to challenge their insurance coverage (e.g., the user indicates they have coverage for aligner treatment and have lifetime max remaining but they are nonetheless denied coverage), the medical provider may review the payor's X12 270 request and response to confirm the payor's coverage. Once the X12 270 request has been generated, the request is communicated to the insurance network 1324 at operation 1322 via an Application Programming Interface (e.g., via an API configured to handle an X12 EDI data exchange). At operation 1332, the pricing calculator 10 determines whether there has been an error response from the insurance network 1324. If an error is observed at operation 1332, then the pricing calculator 10 sets the X12 271 response to the X12 270 request to be a generic unknown X12 271 before proceeding to operation 1340. If no error response is observed at operation 1332, then the process 1300 continues directly to operation 1340. At operation 1340, the X12 271 response is parsed into an object structure. Typically, when the insurance network 1324 outputs an X12 271 response, it is of a complex format. Therefore, the pricing calculator 10 standardizes the X12 271 response into simpler and/or standardized format at operation 1340.

At operation 1342, the pricing calculator 10 retrieves the aligner purchase information for a particular user. In some embodiments, the aligner purchase information can include the price of the aligner purchase and the procedure code for the aligner purchase. In some embodiments, this purchase may be obtained from a medical provider's network. The medical provider's network may include the medical provider's website and all its related applications and pages. At operation 1344, the pricing calculator 10 determines a specific rules engine to use based on the particular rules associated with an instant payor and/or a medical provider. The rules engine is specific because it is related to a particular payor of a user seeking a particular medical procedure (e.g., a particular aligner purchase) covered by the payor. In some embodiments, the specific rules engine includes rules provided by the insurance policy, the medical services provider, or a combination of both. For example, the insurance policy may include a rule that the insurance network covers orthodontic procedures for minors at 100% and adult orthodontic procedures at only 50%. Additionally, the medical provider may have a rule that they do not provide medical services or procedures to children under a certain age. As another example, the medical provider may have rules regarding the type of insurance plans they accept, whether they accept users without insurance, whether they accept users without orthodontic coverage, whether they accept users with a waiting period in their insurance policy, and ensuring that the user's insurance policy is valid. At operation 1346, the pricing calculator 10 determines if a rules engine has been found. If a rules engine is not found, the user's information is sent to a third party customer handling software 1328 and the process 1300 ends at operation 1336. If a rules engine is found, then the pricing calculator 10 runs the specific rules found in the rules engine at operation 1348. In some embodiments, each rules engine can individually determine which rules to run. This may be accomplished via a plugin system in which specific rules are introduced into the rules engine. The plugin system uses the built in dependency injection system within .NET core software to inject one or more rules class files into the rules engine based on the specific payor. For example, multiple rules class files can be injected into the rules engine to enhance a specific instance of the payor's ability to determine medical coverage for aligners.

Once the rules engine finishes running at operation 1348, the pricing calculator 10 retrieves specific information about the user's coverage at operations 1350-1360. In some embodiments, at operation 1350, the pricing calculator 10 determines the user's deductible from the X12 271 response at operation 1350. At operation 1352, the pricing calculator 10 determines what portion of the user's lifetime max is remaining. At operation 1354, the pricing calculator 10 determines the user's co-insurance percent. At operation 1356, the pricing calculator 10 determines whether the user has already purchased the medical service or procedure. If the user has already purchased the medical service or procedure, then the pricing calculator 10 determines if the purchase was made between the plan dates of when the insurance policy was valid and if the purchase was made with an in-network provider at operations 1358 and 1360 respectively. The process 1300 then ends at operation 1362. If the user has not already purchased the medical service or procedure, then the process proceeds to operation 1362 where process 1300 ends and process 1400 begins.

Referring now to FIGS. 14A-14D, a process 1400 for determining an obligation estimation for a medical procedure is shown, according to an exemplary embodiment. The process 1400 can be performed by one or more processors, such as processor 14. As mentioned above, the process 1400 picks up where the process 1300 ended. Whereas the process 1300 determines coverage for a particular medical procedure, the process 1400 determines the obligation (e.g., total cost) to a user for the particular medical procedure, taking into account the user's coverage determined in process 1300. The process 1400 begins similarly to process 1300 by pulling a configuration for a user at operation 1404 from the configuration table 1414. If the user has a configuration at operation 1406, then process 1400 proceeds to operation 1406. At operation 1406, the pricing calculator 10 determines if the user has already purchased the medical procedure. If the user has already purchased the medical procedure, then the pricing calculator 10 determines if the purchase date was on or before the user's enrollment date to their insurance plan at operation 1410. If the purchase date was on or before the user's enrollment date to their insurance plan, the pricing calculator 10 determines if the purchase date falls within the insurance plan dates at operation 1412. If the purchase date falls within the insurance plan dates, then the process 1400 proceeds to operation 1450 shown in FIG. 14B. If the conditional at operation 1410 is negative (i.e., purchase date was before enrollment date), then the process 1400 instead proceeds to operation 1416.

At operation 1416, the pricing calculator 10 determines whether the user has an active dental insurance policy. If the user does not have an active dental insurance policy, then the pricing calculator 10 determines that the user is out of network and does not have any coverage at operation 1430. Then the pricing calculator 10 sets the lead status as complete at operation 1432 and updates the eligibility aggregate (i.e. the amount of the procedure covered by insurance) at operation 1438. The lead status refers to a state of a potential customer's purchase. For example, once a user indicates that they would be interested in purchasing a service or device (e.g., by clicking get started buttons 108 or 708), the pricing calculator 10 determines that there is a “new lead”. The new lead may have one or more statuses. For example, a new lead status may be “In Progress” which means the new lead is still being processed by the pricing calculator 10. In another example the new status may be “Complete — Pending Discount Code” that refers to a state where the pricing calculator has delivered results to the customer on screen, but requires the manual creation of the actual discount code. In another example the new status may be “Complete — Pending approval” that refers to a state where a discount has been manually created and applied and is pending final review by an inspector. As a final example, the new lead status may be “Complete” that refers to a state where the new lead has been fulfilled and delivered to the customer. In some embodiments, the eligibility aggregate may be saved in a database 1442. In some embodiments, the obligation estimation circuit 20 can pull this eligibility aggregate from the database in order to provide a total cost estimation to a user. In addition to storing the eligibility aggregate to the database 1442, the eligibility aggregate is also sent to a third party customer handling software at operation 1440. If the user does have an active dental policy at operation 1416, then pricing calculator 10 evaluates a number of conditionals at operations 1418-1426. More specifically, the conditional at operation 1418 determines if the user has orthodontic coverage. The conditional at operation 1420 determines if the user has out of network coverage. The conditional at operation 1422 determines how much funds the user has before reaching their lifetime maximum benefit. The conditional at operation 1424 determines if the user is within an appropriate age limit. The conditional at operation 1426 determines if the user is enrolled in a commercial plan. If the pricing calculator 10 determines that any of these conditionals is negative, then the process 1400 proceeds to operation 1430, which is described above.

In another embodiment, the rules engine may have already determined the outcome of one or more conditional statements at operations 1418-1426. In such an embodiment, the process 1400 can be improved to run more efficiently (e.g., faster and consuming less power) by being rearranged so that these conditionals would not be again evaluated, thus saving computer processing power and time. For example, the rules engine may have the age plugin enabled that confirms that the user is already within a certain age limit. Since the user's age has already been confirmed, the computer can save processing time and power by not determining the conditional at operation 1424. In this case, operations 1418-1426 would be replaced with the operations 1418A-1426A shown in FIG. 14C, which demonstrate the more streamlined process. In another example, the rules engine may have the orthodontic coverage plugin enabled that confirms that the user has orthodontic coverage. Since it is already confirmed that the user has orthodontic coverage, the computer can save processing time and power by not determining the conditional at operation 1418. In this case, operations 1418-1426 would be replaced with the operations 1418B-1426B shown in FIG. 14D that demonstrate the more streamlined process. If all these conditionals are positive, then the pricing calculator 10 determines if any nulls were returned for the conditionals at operations 1418-1426. If a null was returned at operations 1418-1426, then the pricing calculator 10 is unable to verify the user's coverage and sets the results to “unable to verify.” Then the process 1400 proceeds to operation 1432 where the pricing calculator 10 sets the lead status to “new lead”. A new lead refers to when the pricing calculator 10 fails to determine a payor benefit and obligation estimation. This new lead may be provided to the third party customer handling software at operation 1440, which may in turn trigger an agent to act. At operations 1434 and 1436 the discount is set to 0% which means the user must cover 100% of the costs of the medical procedure. Then the process 1400 continues to operation 1438, which is described above. If no nulls were returned for the conditionals at operations 1418-1426, then the pricing calculator 10 determines if the user has already purchase the medical procedure. If the user has not already purchased the medical procedure, then the pricing calculator 10 sets the result to out of network pre-purchase coverage. If the user has already purchased the medical procedure, then the pricing calculator 10 sets the result to out of network post-purchase coverage. In this case, the user has an active dental policy with some coverage for the medical procedure but is not determined to be “in-network”. Then the process proceeds to operation 1500 shown in FIG. 14B.

At operation 1500, the pricing calculator 10 determines if the user is verified. If the user is not verified, then the pricing calculator 10 sets the lead status to instant—verify at operation 1499. The verification process may require a medical provider and/or revenue cycle management agent to manually compare the patient's coverage eligibility as determined by the pricing calculator 10. If the user is verified, then the pricing calculator 10 sets the lead status to complete pending discount code at operation 1474. The “complete pending discount code” lead status refers to a state where the pricing calculator 10 has delivered results to the customer on screen, but requires the manual creation of the actual discount code based on the insurance coverage amount and the discount calculated as described above. The discount code may be created by the revenue cycle management agent. In some embodiments, the “complete pending discount code” lead status triggers the creation of the discount, either automatically or by the revenue cycle management agent. Regardless of whether the conditional at operation 1500 is positive or negative, the process 1400 proceeds to the conditional at operation 1476 which determines whether only a discount will be applied to the user's purchase. For example, the user may be eligible for only a discount if the user is in network but does not have any benefit coverage (e.g., the user has dental coverage but not orthodontics coverage). For example, if the status outcome is set to INNCoverageAndDiscountPrePurchase at 1468 or INNCoverageAndDiscountPostPurchase at 1470, the user is eligible for the full discount plus insurance cost. In another example, if the status outcome is set to InNetworkDiscountOnlyPostPurchase at 1472 or InNetworkDiscountOnlyPrePurchase at 1464, then the user is only eligible for the in network discount. If the user is only eligible for a discount, then the pricing calculator sets the user's discount to a non-orthodontic coverage discount. If the user is eligible to more than a discount, then the pricing calculator 10 sets the deductible to the user's deductible at 1480. The process 1400 then proceeds to calculate the total discount for the user at operations 1484 to 1498. At operation 1482, the pricing calculator 10 sets the discount to the network discount. Then at operation 1484, the pricing calculator 10 sets the net product price (NPP) of the medical procedure equal to the gross product price (GPP) minus the in network discount (IND) determined at operation 1482. Then at operation 1486, the pricing calculator 10 sets the net product price less deductible (NPPLD) to equal the NPP minus the deductible. Then at operation 1488, the pricing calculator 10 sets the max coverage (MIC) equal NPPLD multiplied by the co-insurance percent. At operation 1490, the pricing calculator 10 determines if the MIC is greater that the user's lifetime remaining coverage. If the MIC is greater than the user's lifetime remaining coverage, then the pricing calculator 10 sets the coverage with lifetime max (ICWLTM) equal to the user's lifetime remaining coverage at 1492. If the MIC is less than the user's lifetime remaining coverage, then the pricing calculator 10 sets the ICWLTM equal to the MIC at 1494. Then at operation 1496, the pricing calculator 10 sets the customer's out of pocket (COOP) equal to NPP minus ICWLTM. This is the total cost estimation that is provided to the user at interface portion 614 in FIG. 6. Then at operation 1498, the pricing calculator 10 sets the discount code amount equal to ICWLTM plus the discount determined at either operation 1482 or 1478. Then the process proceeds back to operation 1438 which is explained above.

As explained above, if the conditional at operation 1408 is negative (i.e., the user has not already purchased the medical procedure) or the conditional at operation 1412 (i.e., the purchase dates fall within the plan dates), then the process 1400 proceeds to operation 1450. At operation 1450, the pricing calculator 10 determines if the user has an active dental policy. If the user does not have an active dental policy, then the process 1400 proceeds to operation 1504 where the pricing calculator 10 sets the user's coverage result to in network, no active dental plan. Then at operation 1502, the pricing calculator 10 sets the user's discount to zero percent and proceeds to operation 1438, which is described in more detail above. If the user does have an active dental insurance policy at operation 1450, the pricing calculator 10 evaluates a number of conditionals at operations 1452-1458. More specifically, the conditional at operation 1452 determines if the user has a commercial plan. The conditional at operation 1454 determines if the user has orthodontic coverage. The conditional at operation 1456 determines how much funds the user has before reaching their lifetime maximum benefit. The conditional at operation 1458 determines if the customer is within an appropriate age limit as determined by the rules engine process shown described with respect to FIG. 13B. If any of the conditionals are negative, then the process proceeds to operation 1462 where the pricing calculator 10 determines whether the medical procedure has already been purchased. If the medical procedure has already been purchased, then the process proceeds to set the user's coverage result to in network discount only post purchase at operation 1472. Then the process proceeds to operation 1500, which is explained above. If the medical procedure has not already been purchased, then the process proceeds to set the user's coverage result to in network discount only pre-purchase at operation 1464. Then the process proceeds to operation 1500, which is explained above. If all the conditionals at operations 1452-1458 are positive, then the pricing calculator 10 determines if any nulls were returned for the conditionals at operations 1452-1458. If a null was returned at any of operations 1452-1458, then the process proceeds to operation 1430, which is explained above.

If no nulls were returned at operations 1452-1458, then the process proceeds to operation 1466 where the pricing calculator 10 determines whether the medical procedure has already been purchased. If the medical procedure has already been purchased, then the process proceeds to set the user's coverage result to in network coverage and discount post purchase at operation 1470. Then the process proceeds to operation 1500, which is explained above. If the medical procedure has not already been purchased, then the process proceeds to set the user's coverage result to in network coverage and discount pre-purchase at operation 1468. Then the process proceeds to operation 1500, which is explained above.

Though the operations detailed above with respect to processes 1300 and 1400 are described in a certain order, these descriptions are only meant to be exemplary. The operations of process 1300 and 1400 may be performed in different orders than they are described herein. Additionally, in some embodiments, one or more of the operations of process 1300 and 1400 may be omitted from being performed in process 1300 and 1400. In some embodiments, one or more processors, such as processor 14, performing process 1300 or 1400 can omit one or more of the operations of process 1300 or 1400, thereby saving computing resources by not expending unnecessary computing resources, and performing the overall process 1300 or 1400 more quickly and efficiently.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

It is noted that terms such as “approximately,” “substantially,” “about,” or the like may be construed, in various embodiments, to allow for insubstantial or otherwise acceptable deviations from specific values. In various embodiments, deviations of 20 percent may be considered insubstantial deviations, while in certain embodiments, deviations of 15 percent may be considered insubstantial deviations, and in other embodiments, deviations of 10 percent may be considered insubstantial deviations, and in some embodiments, deviations of 5 percent may be considered insubstantial deviations. In various embodiments, deviations may be acceptable when they achieve the intended results or advantages, or are otherwise consistent with the spirit or nature of the embodiments.

Example computing systems and devices may include one or more processing units each with one or more processors, one or more memory units each with one or more memory devices, and one or more system buses that couple various components including memory units to processing units. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated modules, units, and/or engines, including processor instructions and related data (e.g., database components, object code components, script components, etc.), in accordance with the example embodiments described herein.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

1. A system comprising:
a processor and a non-transitory computer-readable medium comprising computer-readable instructions stored thereon that when executed by the processor cause the processor to:
collect a first data set associated with a user by a user interface of a user device;
collect a second data set;
generate a new data structure using the first data set and the second data set, the new data structure comprising an electronic data interchange request for a third data set;
transmit the electronic data interchange request to a remote third party computing system;
receive an electronic data interchange response containing the third data set in response to the transmitted electronic data interchange request;
determine one or more rules associated with at least one of a manufacturer of a device or a service provider;
determine one or more rules associated with a benefit provider;
generate a rules engine comprising the one or more rules associated with at least one of the manufacturer or the service provider and the one or more rules associated with the benefit provider that, when enabled, evaluate the third data set based on the first data set and the second data set, wherein the rules engine includes an orthodontic coverage plugin configured to determine whether the user is eligible for orthodontic coverage;
run the rules engine to evaluate the third data set using data from at least one of the first data set or the second data set as an input;
determine an eligibility status of the user associated with the first data set for at least one of a product or a service provided by at least one of the manufacturer or the service provider based on the evaluation of the third data set by the rules engine;
determine an insurance coverage parameter for the user;
determine a discount parameter for the user associated with the first data set;
determine an obligation parameter for the user based on the insurance coverage parameter, the discount parameter, and the third data set; and
display the insurance coverage parameter, the discount parameter, and the obligation parameter on the user interface of the user device.

2. The system of claim 1, wherein the rules engine enables or disables one or more rules to evaluate the third data set based on a parameter of the third data set.

3. The system of claim 1, wherein the rules engine enables or disables one or more rules to evaluate the third data set based on the rules associated with at least one of the manufacturer or the service provider.

4. The system of claim 1, wherein determining the insurance coverage parameter comprises disabling the one or more rules for determining a conditional based on determining that the generated rules engine includes an enabled plugin configured to determine the conditional.

5. The system of claim 1, wherein the electronic data interchange request is a X12 270 electronic data interchange request, and wherein the electronic data interchange response is a X12 271 electronic data interchange response.

6. The system of claim 1, wherein the first data set includes personal information of the user and the second data set includes at least one of a member ID of the user or a policy number of the user, and wherein the instructions when executed cause the processor to parse the third data set to convert the third data set to a standardized format.

7. The system of claim 1, wherein the user is at least one of a policy holder of a policy for the insurance coverage parameter, a beneficiary of the policy, a recipient of treatment related to the insurance coverage parameter, or a third party.

8. A method comprising:
collecting a first data set associated with a user by a user interface of a user device;
collecting a second data set;
generating a new data structure using the first data set and the second data set, the new data structure comprising an electronic data interchange request for a third data set;
transmitting the electronic data interchange request to a remote third party computing system;
receiving an electronic data interchange response containing the third data set in response to the transmitted electronic data interchange request;
determining one or more rules associated with at least one of a manufacturer of a device or a service provider;
determining one or more rules associated with a benefit provider;
generating a rules engine comprising the one or more rules associated with at least one of the manufacturer or the service provider and the one or more rules associated with the benefit provider that, when enabled, evaluate the third data set based on the first data set and the second data set, wherein the rules engine includes an orthodontic coverage plugin configured to determine whether the user is eligible for orthodontic coverage;
running the rules engine to evaluate the third data set using data from at least one of the first data set or the second data set as an input;
determining an eligibility status of the user associated with the first data set for at least one of a product or a service provided by at least one of the manufacturer or the service provider based on the evaluation of the third data set by the rules engine;
determining an insurance coverage parameter for the user;
determining a discount parameter for the user associated with the first data set;
determining an obligation parameter for the user based on the insurance coverage parameter, the discount parameter, and the third data set; and
displaying the insurance coverage parameter, the discount parameter, and the obligation parameter on the user interface of the user device.

9. The method of claim 8, wherein the rules engine enables or disables one or more rules to evaluate the third data set based on a parameter of the third data set.

10. The method of claim 8, wherein the rules engine enables or disables one or more rules to evaluate the third data set based on the rules associated with at least one of the manufacturer or the service provider.

11. The method of claim 8, wherein determining the insurance coverage parameter comprises disabling the one or more rules for determining a conditional based on determining that the generated rules engine includes an enabled plugin configured to determine the conditional.

12. The method of claim 8, wherein the electronic data interchange request is a X12 270 electronic data interchange request, and wherein the electronic data interchange response is a X12 271 electronic data interchange response.

13. The method of claim 12, wherein the method further comprises sending the X12 270 electronic data interchange request to the remote third party computing system via an Application Programming Interface.

14. The method of claim 8, wherein the first data set includes personal information of the user and the second data set includes at least one of a member ID of the user or a policy number of the user, the method further comprising parsing the third data set to convert the third data set to a standardized format.

15. The method of claim 8, wherein the user is at least one of a policy holder of a policy for the insurance coverage parameter, a beneficiary of the policy, a recipient of treatment related to the insurance coverage parameter, or a third party.

16. A non-transitory computer-readable media comprising computer-readable instructions stored thereon that when executed by a processor cause the processor to:
collect a first data set;
collect a second data set;
generate a new data structure using the first data set and the second data set, the new data structure comprising an electronic data interchange request for a third data set;
transmit the electronic data interchange request to a remote third party computing system;
receive an electronic data interchange response containing the third data set in response to the transmitted electronic data interchange request;
determine an insurance coverage parameter for a user associated with the first data set based on the third data set using a rules engine;
identify a plurality of users that share at least one commonality with the user based on the first data set of the user;
generate a message based on the insurance coverage parameter for the user, the message including an indication of a potential insurance coverage parameter for the plurality of users; and
provide the message to user devices associated with the plurality of users that share the at least one commonality with the user.

17. The non-transitory computer-readable media of claim 16, wherein the message is digitally distributed on a website visited by at least one of the plurality of users.

18. The non-transitory computer-readable media of claim 16, wherein the computer-readable instructions cause the processor to determine that the plurality of users likely are eligible to receive the same insurance coverage parameter based on the at least one commonality.

19. The non-transitory computer-readable media of claim 16, wherein the message indicates that the plurality of users may be eligible to receive the same insurance coverage parameter.

20. The non-transitory computer-readable media of claim 16, wherein the computer-readable instructions cause the processor to generate and distribute the message based on the identified commonality being that the plurality of users and the user have the same employer.
